(19) United States
(12) Patent Application Publication
(10) Pub. No.: US 2020/0357484 A1
(43) Pub. Date: Nov. 12, 2020

(54) METHOD FOR SIMULTANEOUS MULTIVARIATE FEATURE SELECTION, FEATURE GENERATION, AND SAMPLE CLUSTERING

(71) Applicant: KONINKLIJKE PHILIPS N.V., EINDHOVEN (NL)

(72) Inventors: Kostyantyn Volyanskyy, Larchmont, NY (US); Nevenka Dimitrova, Pelham Manor, NY (US)

(21) Appl. No.: 16/762,371

(22) PCT Filed: Oct. 23, 2018
(86) PCT No.: PCT/EP2018/078941

§ 371 Date: May 7, 2020

Related U.S. Application Data
(60) Provisional application No. 62/583,034, filed on Nov. 8, 2017.

(51) Int. Cl. G16B 40/00; G16B 50/00; G16B 40/30

(52) U.S. Cl. CPC G16B 40/00; G16B 40/30; G16B 50/00

(57) ABSTRACT

A genomic/proteomic test synthesis method includes receiving a genomic/proteomic data set (12) comprising samples corresponding to persons with each sample including values of features of a set of features derived from genomic/proteomic data for the corresponding person. For each feature, univariate analysis (30) is performed to generate a sample density versus feature value data set for the feature, for example represented as a kernel density estimate (KDE) (52). Multivariate analysis (32, 34) is performed on the features using the KDEs to generate a set of discriminative features (36, 38). In one example, the multivariate analysis (32) uses energy spectral density (ESD) characteristics of the KDEs. In another example, the multivariate analysis (34) uses peak location characteristics of the KDEs.

### FIELD

The following relates generally to the clinical testing arts, genomic testing arts, proteomic testing arts, and related arts.

### BACKGROUND

Genomic and proteomic testing is increasingly applied as tools for diagnosing and typing cancers, determining pathogen strains, and other clinical tasks. These techniques are capable of producing vast quantities of data.

Genomic testing may employ next-generation sequencing (NGS) to acquire a whole genome sequence (WGS), a whole exome sequence (WES, including only protein-encoding exons), RNA sequences, or so forth. In a typical NGS workflow, a tissue sample from a cancerous tumor or other tissue of interest is drawn via a biopsy or other interventional procedure. Wet lab processing is used to extract, purify or otherwise prepare deoxyribonucleic acid (DNA) from the sample, followed by target enrichment (e.g. for WES), polymerase chain reaction (PCR) amplification, and/or other sample processing. The prepared sample is loaded into a NGS genetic sequencer that generates unaligned DNA sequence fragment reads (data representations of base sequences of DNA fragments) which may for example be stored as FASTQ data files. The unaligned reads are aligned with a reference DNA sequence using suitable data processing such as a Burrows-Wheeler Alignment (BWA) tool followed by SAMtools to align longer sequences. The aligned DNA sequence (e.g. WGS or WES sequence) is stored as a Sequence Alignment/Map (SAM) or Binary Alignment Map (BAM) or similar-type file. Variant calling software may be applied to identify genetic variants such as single nucleotide polymorphism (SNP) or single nucleotide variant (SNV) variants, base modification variants (e.g. methylation), extra or missing bases (inserts or deletes, i.e. indels), copy number variations (CNVs), or so forth. A list of genetic variants may be stored as a standard variant calls file (VCF) or the like.

Proteomic data may be acquired from a tissue sample using laboratory tools such as mass spectroscopy or microarray or protein chip analysis. For example, cells of a microarray are designed to interrogate specific proteins, and the outputs of the cells represent protein concentrations quantifying gene expression levels for corresponding genes. Mass spectroscopy similarly quantifies concentrations of resolved proteins in the sample. As with NGS, large quantities of data can be generated. Combining genomic and proteomic analyses can in principle provide synergistic information.

However, extracting clinically useful information from genomic or proteomic data sets is challenging. In a supervised learning approach, samples in the form of WGS, gene expression data or the like for various patients is analyzed. In a supervised approach the samples (i.e. patients) are labeled as to whether they have the clinical condition of interest (e.g. the type of cancer). In such cases, the analysis amounts to identifying correlations between various features of the genomic/proteomic data (where a feature may be a genetic variant, a certain expression level bin, or so forth) and presence/absence of the clinical condition of interest. This can be challenging when the genomic/proteomic data set contains tens of thousands of features.

Supervised learning is restricted to samples that are labeled as to the clinical condition of interest, and cannot leverage unsupervised data, that is, samples which are not labeled as to presence/absence of the clinical condition of interest. Thus, supervised learning of genomic and/or proteomic tests cannot leverage data sets without the appropriate clinical labeling. On the other hand, unsupervised learning techniques employ clustering or the like to group together similar samples, without regard to clinical labeling. These clusters can then be compared with any available labeled data to derive useful information from the unlabeled data. However, unsupervised learning of useful clinical tests in the absence of clinical labeling of (at least most) samples is even more challenging than supervised learning.

To address the dimensionality challenge and associated issues, techniques such as deep learning auto-encoders have been used to reduce the dimensions of the feature space and compress the data structure while minimizing the data content loss. However, the structure of the auto-encoder needs to be defined in advance, and optimization results as well as data compression depend strongly on this pre-defined structure; yet, there is little guidance available to the test developer as to how to optimally pick such a structure.

The following discloses a new and improved systems and methods.

### SUMMARY

In one disclosed aspect, a genomic/proteomic test synthesis device comprises a computer and a non-transitory storage medium that stores instructions readable and executable by the computer to perform a genomic/proteomic test synthesis method. That method includes: receiving a genomic/proteomic data set comprising samples corresponding to persons with each sample including values of features of a set of features derived from genomic/proteomic data for the corresponding person; for each feature, generating a kernel density estimate (KDE) of sample density versus feature value for the feature; and performing multivariate analysis on the features using the KDEs to generate a set of discriminative features.

In another disclosed aspect, a non-transitory storage medium stores instructions readable and executable by an electronic processor to perform a genomic/proteomic test synthesis method comprising: receiving a genomic/proteomic data set comprising samples corresponding to persons with each sample including values of features of a set of features derived from genomic/proteomic data for the corresponding person; for each feature, performing univariate analysis on the values of the feature for the samples of the genomic/proteomic data set to generate a sample density versus feature value data set for the feature; and performing multivariate analysis on the features using the sample density versus feature value data sets to generate at least one set of discriminative features.

In another disclosed aspect, a genomic/proteomic test synthesis method is disclosed. A genomic/proteomic data set is received at a computer. The data set comprises samples corresponding to persons with each sample including values of features of a set of features derived from genomic/proteomic data for the corresponding person. For each feature and using the computer, univariate analysis is performed on the values of the feature for the samples of the genomic/proteomic data set to generate a sample density versus feature value data set for the feature. Using the computer, multivariate analysis is performed on the features using the sample density versus feature value data sets to generate at least one set of discriminative features.

One advantage resides in providing more robust feature selection for synthesis of a genomic/proteomic test.

Another advantage resides in providing more efficient synthesis of a genomic/proteomic test.

Another advantage resides in providing more computationally efficient detection of the most discriminative features for use in synthesis of a genomic/proteomic test.

Another advantage resides in providing selection of the most discriminative features for use in synthesis of a genomic/proteomic test that is effective to detect single features that are highly discriminative.

Another advantage resides in providing one or more of the foregoing benefits without the need for a labeled (or fully labeled) samples data set.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention. In drawings presenting log or service call data, certain identifying information has been redacted by use of superimposed redaction boxes.

FIG. 1 diagrammatically illustrates a genomic/proteomic testing system including a genomic/proteomic test synthesis system.

FIGS. 2, 3, 4 and 5 diagrammatically show processing embodiments of the genomic/proteomic testing system of FIG. 1.

FIGS. 6 and 7 plot univariate analysis results for two illustrative gene expression level features suitably produced by the genomic/proteomic testing system of FIG. 1

### DETAILED DESCRIPTION

Some approaches for genomic/proteomic test synthesis disclosed herein proceed in two stages. First, univariate feature pre-selection is performed, since there is a possibility of even a single feature providing important characterization of a dataset. Next the process iterates over features ranked by the analysis results of the first step and detects associated sample clustering while doing forward selection and non-linear transformation of features. Clustering characteristics such as connectedness, homogeneity, and/or so forth may be assessed to include or exclude certain features from further iterations. One or more sets of discriminative features are obtained, and associated sample clusters that characterize the data set based on the chosen criteria. For clinical applications the discriminative features are linked with sample groups defined by clinical variables to provide analytic solutions for predictive diagnostics, and biomarker detection.

The disclosed approaches provide efficient feature selection by way of unsupervised learning, and various embodiments exhibit advantages such as one or more of the following: improved characterization of an arbitrary dataset; improved capturing of important features; and/or improved performance of predictive modelling schemes.

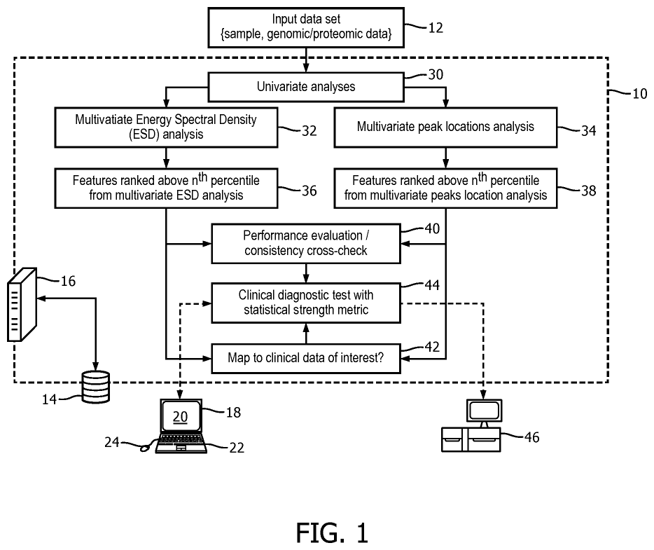

With reference to FIG. 1, an illustrative genomic/proteomic test synthesis device 10 operates on an input data set 12 comprising {sample, genomic/proteomic data}, i.e. a genomic/proteomic data set comprising samples corresponding to persons with each sample including values of features of a set of features derived from genomic/proteomic data for the corresponding person. As used herein, the phrases “genomic/proteomic test”, “genomic/proteomic data set”, and similar phraseology is intended to encompass tests, data sets, et cetera that operate on or include only genomic data; or that operate on or include only proteomic data; or that operate on or include both genomic data and proteomic data. Genomic data encompasses information from genetic sequences or information derived from genetic sequences, such as values of specific nucleotides and/or values of genetic variants such as single nucleotide polymorphism (SNP) or single nucleotide variant (SNV) variants, base modification variants (e.g. methylation), extra or missing bases (inserts or deletes, i.e. indels), copy number variations (CNVs), or so forth. Proteomic data encompasses information on protein expression (including RNA transcription), protein concentrations or expression levels in serum samples, and so forth, for example measured using micro arrays, mass spectroscopy, or other suitable laboratory techniques. In the illustrative example, the input data set 12 is provided as a table in which N (N>0) samples are given as rows, and M (M>0) features as columns. In addition, a set of class labels may be provided for all N samples or for some fraction of the N samples. By way of illustration, the input data set 12 may be drawn from standard variant calls file (VCF) or the like for genetic variants, or from FASTQ data files or other raw sequence data files in the case of specific nucleotide values. Proteomic data may be drawn from protein expression levels provided by micro array or mass spectroscopy data or so forth. It is contemplated for a portion of the M features to be derived features, e.g. a binary value indicating the patient corresponding to the sample has some specific combinations of variants. The class labels provide clinical data of interest, such as by way of illustration a label indicating whether the patient/sample has a specific type of cancer, a label indicating the cancer stage, a label indicating the cancer grade, labels indicating demographic information, labels indicating geographical location information, labels indicating lifestyle information such as smoker/nonsmoker, labels indicating clinical information such as age and/or weight, et cetera.

As diagrammatically indicated in FIG. 1, the genomic/proteomic test synthesis device 10 is implemented as a non-transitory storage medium 14 which stores instructions that are readable and executable by a computer or other electronic processor 16, 18 to perform a genomic/proteomic test synthesis method as disclosed herein. The non-transitory storage medium 14 may, by way of non-limiting illustration, comprise a hard disk drive, RAID disk array or other magnetic storage medium; a solid state drive (SSD) or other electronic storage medium, an optical disk or other optical storage medium, various combinations thereof, or so forth. The computer or other electronic processor 16, 18 may be a server computer 16, a desktop computer 18, a plurality of operatively interconnected server and/or desktop computers, optionally connected in an ad hoc fashion forming a cloud computing resource, and/or so forth. The genomic/proteomic test synthesis device 10 may further include a display 20 for presenting results or other information, and one or more user input devices such as an illustrative keyboard 22, mouse 24, touch-sensitive overlay of the display 20 (i.e. the display may be a touchscreen user input device), various combinations thereof, or so forth.

As diagrammatically indicated in FIG. 1, the genomic/proteomic test synthesis method implemented by the device 10 includes performing univariate analyses 30 to generate a sample density versus feature value data set for the feature. This may be in the form of a histogram for each feature that stores the number of samples in each feature value bin. A disadvantage of histogram analysis is that it produces discontinuous data with low granularity. In the illustrative embodiment, the univariate analyses 30 produce a kernel density estimate (KDE) of sample density versus feature value for each feature of the M features.

The univariate analyses 30 are followed by one or more multivariate analyses 32, 34, which in the illustrative embodiment include: (1) a multivariate energy spectral density (ESD) analysis 32 producing a top-ranked set of features 36, e.g. ranked above some n^{th} percentile of the M features; and (2) a multivariate peak locations analysis 34 producing a top-ranked set of features 38, e.g. ranked above some n^{th} percentile of the M features (where a different percentile n is optionally used versus the ESD ranging 36). In the illustrative approaches, clustering of samples is used to assess and rank the features, and clustering performance metrics can then be used in an operation 40 to evaluate performance of the features in discriminating samples from one another. Further, if two or more top-ranked sets of features 36, 38 are generated then the operation 40 can also include a consistency cross-check, e.g. using a rand index comparison.

If clinical data of interest are available in the form of labels annotated to the samples of the data set 12, then the top-ranked features 36, 38 are also mapped to the clinical data of interest in an operation 42. This allows for identification of the most discriminative features (or combination of features) from the list(s) of top-ranked features 36, 38. For example, the most discriminative feature(s) specifically for distinguishing whether a patient has a particular form of cancer may be more effectively distinguished using the mapped labeling for this cancer type.

The list(s) of top-ranked features 36, 38 along with the statistical information from the statistical performance evaluation(s) 40 and the clinical data mapping 42, are used to generate a clinical diagnostic test 44 with a statistical strength metric indicating how strongly the identified feature or set of features correlates with the test output (which may, for example, be an indication of whether the clinical patient has a certain type of cancer). The genomic/proteomic test 44 synthesized using the device 10 of FIG. 1 is applied in conjunction with genomic and/or proteomic data acquired of a clinical patient using a suitable device such as an illustrative gene sequencer 46 for acquiring genomic data, or a micro array or mass spectrometer (not shown) for acquiring proteomic data. In some embodiments, the generated clinical diagnostic test is coded into diagnostics built into the gene sequencer 46 (e.g. code executed by a computer or other electronic processor of the gene sequencer 46 to apply the test 44 to acquired genomic data or variants extracted from such genomic data) or into a computer that processes genomic/proteomic data acquired of a patient.

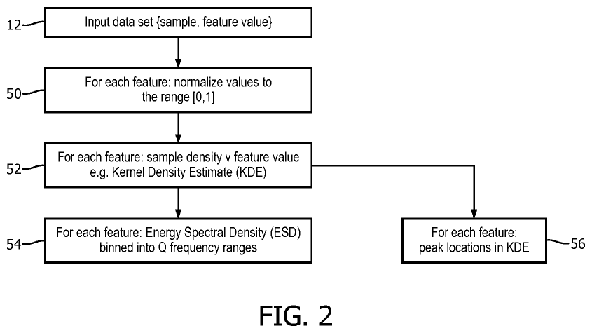

With continuing reference to FIG. 1 and with further reference to FIGS. 2-5, some illustrative examples of various operations of the genomic/proteomic test synthesis method implemented by the device 10 are described.

With reference to FIGS. 1 and 2, the univariate analyses 30 are in one embodiment implemented as a kernel density estimate (KDE) of the sample density versus feature value for each feature of the M features as follows. In the following, each feature is denoted as Fⱼ, j=1, . . . , M (where again M is the number of features) and each feature is represented as a vector or ordered set Fⱼ={V₁ⱼ, . . . , V_{Nj}} where Vᵢⱼ is the value of the feature Fⱼ for the sample indexed by i. In an operation 50, the feature values are normalized to the range [0,1] according to:

$${\{ {V}_{ij} \}}^{norm} ← \frac{{V}_{ij} - {V}_{j}^{min}}{{V}_{j}^{max} - {V}_{j}^{min}} \qquad ( 1 )$$

where Vⱼ^{max}=max{V₁ⱼ, . . . , V_{Nj}} is the largest value of the feature, and Vⱼ^{min}=min{V₁ⱼ, . . . , V_{Nj}} is the smallest value of the feature. For all operations subsequent to the normalization operation 50, the normalized values {Vᵢⱼ}^{norm} is indicated simply as Vᵢⱼ for simplicity of notation herein.

The kernel density estimate (KDE) 52 is then computed according to:

$$KDE ( {F}_{j} ) ≡ {KDE}_{j} ( x ) = \frac{1}{Nh} {\sum }_{i = 1}^{N} K ( \frac{x - {V}_{ij}}{h} ) \qquad ( 2 )$$

where KDEⱼ(x) is the KDE for (normalized) feature Fⱼ and is defined over the interval [0,1], K( . . . ) is the kernel function, e.g. a Gaussian kernel may be used in some embodiments, and h is the kernel bandwidth and is chosen to be sufficiently small to provide the desired resolution along the interval [0,1] and sufficiently large to provide smoothing. The kernel density estimate KDEⱼ(x) of Equation (2) is merely one illustrative embodiment of a suitable smoothed sample density versus feature value data set, and other formulations are contemplated.

The sample density versus feature value data set for each feature Fⱼ quantitatively captures the distribution of the value of the feature over the N samples. This can be further summarized in various ways. For example, the (preferably normalized) energy spectral density (ESD) 54 of each KDE 52 may be used. In computing the ESD, the kernel density estimate KDEⱼ(x) is treated as a finite energy time-series signal, and the ESD may be computed as:

$$ESD ( {KDE}_{j} ( x ) ) ≡ {E}_{j} ( f ) = \frac{1}{2 π} {\int }_{- π}^{π} {( {e}^{- 2 π ifx} {KDE}_{j} ( x ) )}^{2} dx \qquad ( 3 )$$

where f denotes frequency in the range [−π,π]. The ESD is binned into Q frequency ranges denoted here as D₁, . . . , D_{Q} over the range [ωₘᵢₙ, ωₘₐₓ] where ωₘᵢₙ=−π and ωₘₐₓ=π. Here, Q is a method parameter allowing flexible evaluation of feature characteristics at various frequency ranges at tuneable resolutions, including the major regions such as low, high and intermediate frequency ranges. In each of the regions D₁, . . . , D_{Q}, the associated energy content is computed from the values of Eⱼ (f) in each given frequency region, that is: E₁ⱼ, . . . , E_{Qj}. These values are normalized to the range [0,1] similarly to Equation (1), i.e.:

$${\{ {E}_{ij} \}}^{norm} ← \frac{{E}_{j} - {E}_{j}^{min}}{{E}_{j}^{max} - {E}_{j}^{min}} \qquad ( 4 )$$

For all operations subsequent to the ESD computation operation 54, the normalized values {Eᵢⱼ}^{norm} is indicated simply as Eᵢⱼ for simplicity of notation herein.

With continuing reference to FIG. 2, the sample density versus feature value data set for each feature Fⱼ may additionally or alternatively be summarized based on the peak locations 56 of the kernel density estimate KDEⱼ(x). In this approach, a second order differential or other peak detector may be used to detect the locations of peaks in KDEⱼ(x).

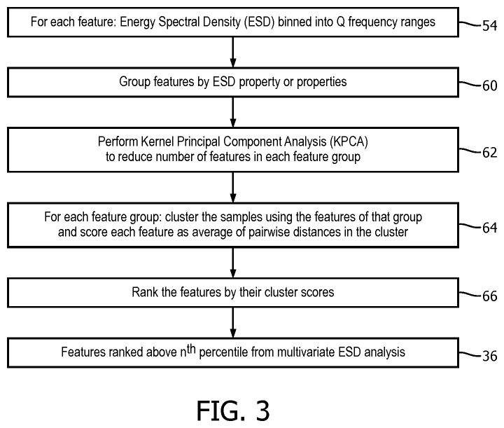

With reference now to FIG. 3, an illustrative example of the multivariate ESD analysis 32 indicated in FIG. 1 is described. The ESD analysis 32 operates on the normalized and binned ESD values 54 denoted here as E₁, . . . , E_{Qj}. In an operation 60, clustering of the features Fⱼ is performed using their (normalized) frequency characteristics Eᵢⱼ, i=1, . . . , Q as their features. In one approach, this clustering groups the features into low, intermediate, and high frequency features as three separate groups. More generally, the grouping of the features Fⱼ based on energy characteristics Eᵢⱼ, i=1, . . . , Q can employ any clustering or grouping scheme, e.g. hierarchical or fuzzy clustering may be used. The output of the operation 60 is a set of feature groups, e.g. a low frequency feature group, an intermediate frequency feature group, and a high frequency feature group in the example.

Optionally, in an operation 62, for each of the feature groups kernel principal component analysis (KPCA) is applied to nonlinearly transform features and identify number of major principal components capturing variance above a chosen threshold (e.g., >=75th percentile).

In an operation 64, clustering of samples is performed using the (optionally KPCA transformed) features of each of the feature groups defined in operation 60 separately, and sample clustering scores are computed for the features as a weighted average of the within-cluster pairwise distances normalized by corresponding cluster sizes. In other words, in the operation 64 for each feature group, clustering of the samples of the data set 12 is performed using the features of that feature group to generate sample clusters for the feature group, and a score is computed for each discriminative feature of the feature group (either original features Fⱼ or KPCA-transformed features, depending on whether operation 62 is performed) on the basis of pairwise distances between samples in the same sample cluster, where the pairwise distances are computed using the values of the discriminative feature for the samples. In an operation 66, the features are ranked by the cluster scores computed in operation 64. The highest-ranked discriminative features 36 are selected using a specific threshold (e.g., 75th percentile or more generally above an n^{th} percentile).

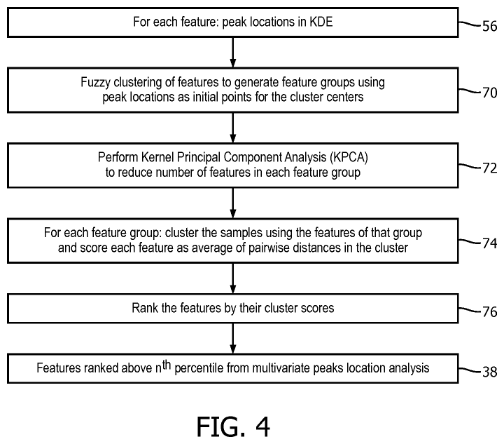

With reference now to FIG. 4, an illustrative example of the multivariate peak locations analysis 34 indicated in FIG. 1 is described. The peak locations analysis 34 operates on the peak locations values 56 for the kernel density estimates KDEⱼ(x) of the features fⱼ from FIG. 2. In an operation 70, fuzzy clustering of features is performed to generate feature groups using the peak locations 56 as initial points for cluster centers. Optionally, in an operation 72, for each of the feature groups kernel principal component analysis (KPCA) is applied to nonlinearly transform features and identify number of major principal components capturing variance above a chosen threshold (e.g., >=75th percentile). KPCA operation 72 is suitably analogous to the KPCA operation 62 of FIG. 3. In an operation 74, clustering of samples is performed using the (optionally KPCA transformed) features of each of the feature groups defined in operation 70 separately, and sample clustering scores are computed for the features as a weighted average of the within-cluster pairwise distances normalized by corresponding cluster sizes. In other words, in the operation 74 for each feature group, clustering of the samples of the data set 12 is performed using the features of that feature group to generate sample clusters for the feature group, and a score is computed for each discriminative feature of the feature group (either original features Fⱼ or KPCA-transformed features, depending on whether operation 72 is performed) on the basis of pairwise distances between samples in the same sample cluster, where the pairwise distances are computed using the values of the discriminative feature for the samples. In an operation 76, the features are ranked by the cluster scores computed in operation 74. The highest-ranked discriminative features 38 are selected using a specific threshold (e.g., 75th percentile or more generally above an n^{th} percentile).

The multivariate analyses 32, 34 using ESD and peak location characteristics, respectively, of the sample density versus feature value data sets 52 are merely illustrative examples. While using both ESD and peak locations in the multivariate analyses 32, 34 is expected to provides synergistic benefits, it is alternatively contemplated to employ only the multivariate analysis 32 using ESD characteristics of the sample density versus feature value data sets 52. As another contemplated alternative, it is contemplated to employ only the multivariate analysis 34 using peak location characteristics of the sample density versus feature value data sets 52. Additional or other multivariate analyses using other characteristics of the sample density versus feature value data sets is also contemplated, such as using discrete Fourier transform characteristics of the sample density versus feature value data sets.

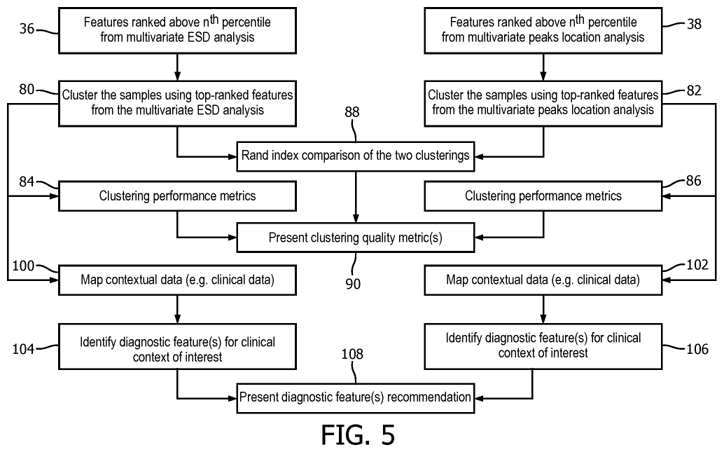

With reference to FIG. 5, an illustrative embodiment of the statistical clustering performance evaluation and optional cross-check 40 and the clinical data mapping 42 are described. In an operation 80, all N samples of the input data set 12 are clustered using the highest-ranked discriminative features 36 chosen using ESD feature grouping. Likewise, in an operation 82 all N samples of the input data set 12 are clustered using the highest-ranked discriminative features 38 chosen using peak locations feature grouping. In an operation 84 clustering performance of the clustering operation 80 is computed, and likewise in an operation 86 clustering performance of the clustering operation 82 is computed. The goal of the clustering performance assessment operations 84, 86 is to determine whether identified clusters are compact and well separated from each other, as desired, or are not well separated. Some non-limiting illustrative metrics for assessing the clustering performance may, for example, include average distance within the cluster, average distance between clusters, normalized within-cluster variance, and/or so forth. In an operation 88, a comparison of the two clusterings 80, 82 is computed, e.g. using a rand index comparison, which is computed as a proportion of agreements of any pair of points ending up in the same cluster, to the total amount of agreements and disagreements. This is equivalent to statistics computed on the confusion matrix. Other methods may work as well such as set matching, and mutual information/entropy-based methods. Based on the clustering performance metrics 84, 86 and the cross-check metric 88, one or more clustering quality metrics are generated and presented on the display 20 in an operation 90.

In an illustrative example of the clinical data mapping operation 42 of FIG. 1, the clinical data labels for the samples of the data set 12 are mapped in respective operations 100, 102 for the respective clusterings 80, 82. Based on the respective mappings, one or more diagnostic features for a clinical context of interest (e.g. patient having a particular type of cancer) are identified from the highest-ranked discriminative features 36 chosen using ESD feature grouping in operation 104; and likewise, one or more diagnostic features for the clinical context of interest are identified from the highest-ranked discriminative features 38 chosen using peak location grouping in operation 106. In an operation 108, the diagnostic feature(s) recommendation is presented on the display 20. This may be combined with the presentation operation 90 to present both the diagnostic features and one or more metrics of how probative these features are, i.e. the strength of correlation between the diagnostic feature(s) and the clinical context of interest. Said another way, the genomic/proteomic test 44 may comprise an association 104, 106 of a clinical condition defined in the mapped clinical data with one or a combination of discriminative features and a statistical strength metric (derived from the clustering quality metrics 90) for the genomic/proteomic test. Since the method identifies the most discriminative features, the presentation operations 90, 108 preferably do not include presenting a result for any feature of the set of features that does not belong to the set of discriminative features 36, 38, thereby increasing efficiency of determination of the clinical diagnostic test 44.

It should be noted that the clinical context labeling of the data set 12 is not used except at the point of performing the mapping operations 100, 102. That is, the selection of the one or more sets of discriminative features 36, 38 entails unsupervised learning that does not rely upon clinical context labeling. Moreover, the mapping operations 100, 102 can map incomplete labeling and perform the diagnostic feature(s) identification 104, 106 with incompletely labeled samples. For example, if only 10% of the samples of the data set 12 are labeled as to a particular cancer type, the labeled 10% of the data can be used to perform the diagnostic feature(s) identification 104, 106, leveraging the unsupervised learning of the one or more sets of discriminative features 36, 38 operating on all 100% of the data set 12 to substantially improve computational efficiency.

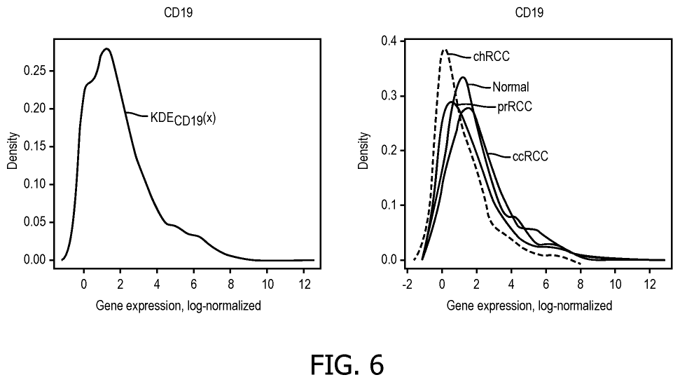

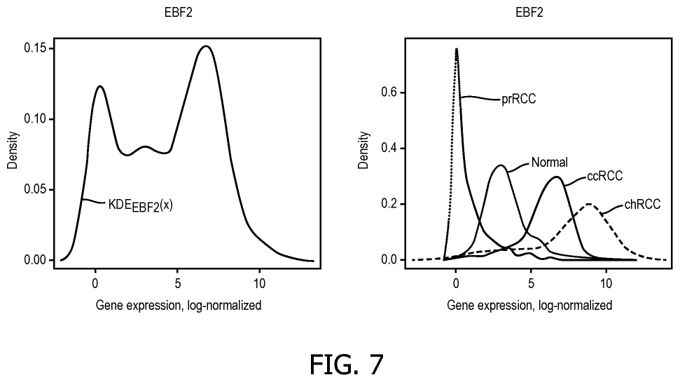

With reference to FIGS. 6 and 7, two examples of features, namely CD19 gene expression (FIG. 6) and EBF2 gene expression (FIG. 7), and their correlation with the clinical contexts of: clear cell renal cell carcinoma (ccRCC), papillary renal cell carcinoma (prRCC), chromophobe renal cell carcinoma (chRCC), and normal tissue (no renal cell carcinoma). The input to the genomic/proteomic test synthesis method in this illustrative example included over 20,000 features (i.e., M>20,000). The left plot of each of FIGS. 6 and 7 shows the kernel density estimate, i.e. KDE_{CD19}(x) in FIG. 6 and KDE_{EBF2}(x) in FIG. 7. An output of the genomic/proteomic test synthesis method is the ranked set of features decided by the potential to represent various distinct sample clusters. In the example of FIGS. 6 and 7, the EBF2 gene expression feature was ranked in the top, while the CD19 gene expression feature was ranked lower; thus, the EBF2 gene expression feature was selected as a discriminative feature whereas CD19 was not selected as a discriminative feature. The righthand plots of FIGS. 6 and 7 show the KDE divided into ccRCC, prRCC, chRCC, and normal groups according to clinical context labeling of the samples. (Said another way, for each clinical group, a KDE is generated of sample density of samples in the clinical group versus discriminative feature value for the discriminative feature). This plot for FIG. 7 illustrates how efficiently the EBF2 gene expression feature differentiates the subtypes and the normal. By contrast, as seen in the right-hand plot of FIG. 6 the CD19 gene expression feature does not differentiate between three RCC subtypes and normal tissue nearly as well as the EBF2 gene expression feature. It is noteworthy that the feature ranking was performed without knowledge of the subtype labeling. In the examples of FIGS. 6 and 7, before the method starts all features (genes) are treated equally. As the method detects the patterns from the KDEs and associated clusterings, some features become ranked higher. The statistical properties of EBF2 showed up as more interesting than those of CD19, and the respective FIGS. 6 and 7 on the right show that this finding has an immediate biological confirmation in that EBF2 is a good indicator on the subtype, while cd19 is not.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A genomic/proteomic test synthesis device comprising:
a computer; and
a non-transitory storage medium storing instructions readable and executable by the computer to perform a genomic/proteomic test synthesis method comprising:
receiving a genomic/proteomic data set comprising samples corresponding to persons with each sample including values of features of a set of features derived from genomic/proteomic data for the corresponding person;
for each feature, generating a kernel density estimate (KDE) of sample density versus feature value for the feature; and
performing multivariate analysis on the features using the KDEs to generate a set of discriminative features.

2. The genomic/proteomic test synthesis device of claim 1 wherein the KDEs employ Gaussian kernels.

3. The genomic/proteomic test synthesis device of claim 1 wherein the performing of multivariate analysis includes:
performing multivariate analysis on the features using energy spectral density (ESD) of the KDEs to generate an ESD-based set of discriminative features.

4. The genomic/proteomic test synthesis device of claim 1 wherein the performing of multivariate analysis includes:
performing multivariate analysis on the features using peak locations in the KDEs to generate a peak locations-based set of discriminative features.

5. The genomic/proteomic test synthesis device of claim 1 wherein the performing of multivariate analysis includes:
grouping features of the set of features into a plurality of feature groups based on characteristics of the KDEs;
for each feature group, performing clustering of the samples using the features of the feature group to generate sample clusters for the feature group;
computing a score for each discriminative feature on the basis of pairwise distances between samples in the same sample cluster wherein the pairwise distances are computed using the values of the discriminative feature for the samples; and
generating the set of discriminative features based on the scores.

6. The genomic/proteomic test synthesis device of claim 1 wherein the performing of multivariate analysis includes:
applying kernel principal component analysis (KPCA) to nonlinearly transform the set of features.

7. The genomic/proteomic test synthesis device of claim 1 further comprising:
a display operatively connected with the computer;
wherein the genomic/proteomic test synthesis method further includes presenting a result for at least one discriminative feature by operations including:
dividing at least a labeled sub-set of the samples of the genomic/proteomic data set into two or more clinical groups on the basis of clinical data of interest for the corresponding persons;
for each clinical group, generating a KDE of sample density of samples in the clinical group versus discriminative feature value for the discriminative feature; and
displaying a graph on the display plotting the KDEs of sample density of samples in the respective clinical groups for the discriminative feature.

8. The genomic/proteomic test synthesis device of claim 7 wherein the presenting does not include presenting a result for any feature of the set of features that does not belong to the set of discriminative features.

9. A non-transitory storage medium storing instructions readable and executable by an electronic processor to perform a genomic/proteomic test synthesis method comprising:
receiving a genomic/proteomic data set comprising samples corresponding to persons with each sample including values of features of a set of features derived from genomic/proteomic data for the corresponding person;
for each feature, performing univariate analysis on the values of the feature for the samples of the genomic/proteomic data set to generate a sample density versus feature value data set for the feature; and
performing multivariate analysis on the features using the sample density versus feature value data sets to generate at least one set of discriminative features.

10. The non-transitory storage medium of claim 9 wherein the performing of univariate analysis includes:
for each feature, computing the sample density versus feature value data set as a kernel density estimate (KDE) of the sample density versus feature value data set.

11. The non-transitory storage medium of claim 9 wherein the performing of multivariate analysis includes:
grouping features of the set of features into a plurality of feature groups based on characteristics of the sample density versus feature value data sets of the features;
for each feature group, performing clustering of the samples using the features of the feature group to generate sample clusters for the feature group;
computing a score for each discriminative feature on the basis of pairwise distances between samples in the same sample cluster wherein the pairwise distances are computed using the values of the discriminative feature for the samples; and
generating the set of discriminative features based on the scores.

12. The non-transitory storage medium of claim 11 wherein the grouping of features of the set of features into the plurality of feature groups includes:
grouping features of the set of features into a plurality of feature groups based on energy spectral density (ESD) characteristics of the sample density versus feature value data sets.

13. The non-transitory storage medium of claim 11 wherein the grouping of features of the set of features into the plurality of feature groups includes:
grouping features of the set of features into a plurality of feature groups based on characteristics comprising peak locations of the sample density versus feature value data sets.

14. The non-transitory storage medium of claim 11 wherein the performing of multivariate analysis further includes:
for each feature group, applying kernel principal component analysis (KPCA) to nonlinearly transform the features of the features group.

15. The non-transitory storage medium of claim 9 wherein the genomic/proteomic test synthesis method further includes:
clustering the samples using the at least one set of discriminative features and computing at least one clustering quality metric for the clustering;
mapping clinical data to the discriminative features; and
displaying a representation of the mapping of the clinical data to the discriminative features of the set of discriminative features.

16. The non-transitory storage medium of claim 15 wherein the genomic/proteomic test synthesis method further includes:
generating a genomic/proteomic test comprising an association of a clinical condition defined in the mapped clinical data with one or a combination of discriminative features and a statistical strength metric derived from the at least one clustering quality metric for the genomic/proteomic test.

17. A genomic/proteomic test synthesis method comprising:
at a computer, receiving a genomic/proteomic data set comprising samples corresponding to persons with each sample including values of features of a set of features derived from genomic/proteomic data for the corresponding person;
for each feature and using the computer, performing univariate analysis on the values of the feature for the samples of the genomic/proteomic data set to generate a sample density versus feature value data set for the feature; and
using the computer, performing multivariate analysis on the features using the sample density versus feature value data sets to generate at least one set of discriminative features.

18. The genomic/proteomic test synthesis method of claim 17 further comprising:
presenting a result for at least one discriminative feature by:
dividing at least a labeled sub-set of the samples of the genomic/proteomic data set into two or more clinical groups on the basis of clinical data of interest for the corresponding persons;
for each clinical group, generating a clinical group sample density versus feature value data set for the feature; and
displaying a graph on the display plotting the clinical group sample density versus feature value data sets for the discriminative feature.

19. The genomic/proteomic test synthesis method of claim 18 wherein the genomic/proteomic test synthesis method does not present a result for any feature of the set of features that does not belong to the set of discriminative features.

20. The genomic/proteomic test synthesis method of claim 17 wherein the performing of multivariate analysis includes:
performing multivariate analysis on the features using energy spectral density (ESD) of the sample density versus feature value data sets to generate an ESD-based set of discriminative features.

21. The genomic/proteomic test synthesis method of claim 17 wherein the performing of multivariate analysis includes:
performing multivariate analysis on the features using peak locations in the sample density versus feature value data sets to generate a peak locations-based set of discriminative features.

22. The genomic/proteomic test synthesis method of claim 17 wherein the univariate analysis comprises generating each sample density versus feature value data set as a kernel density estimate (KDE) of the sample density versus feature values.
